(19) United States
(12) Patent Application Publication
(10) Pub. No.: US 2025/0295631 A1
(43) Pub. Date: Sep. 25, 2025

(54) NOVEL TREATMENT

(71) Applicant: University of Liverpool, Liverpool (GB)

(72) Inventors: Rachel Williams, Liverpool (GB); Kyle Doherty, Liverpool (GB); Hala Dhowre, Liverpool (GB)

(21) Appl. No.: 18/873,070

(22) PCT Filed: Jun. 9, 2023
(86) PCT No.: PCT/GB23/51513

§ 371 Date: Dec. 9, 2024

(30) Foreign Application Priority Data
Jun. 10, 2022 (GB) 2208508.8

(51) Int. Cl. A61K 31/4025; A61K 9/08; A61P 27/02

(52) U.S. Cl. CPC A61K 31/4025; A61K 9/08; A61P 27/02

(57) ABSTRACT

The present invention relates to novel pharmaceutical formulations. More specifically, the present invention relates to novel pharmaceutical formulations that are suitable for administration to the eye. The present invention also relates to the use of these formulations for the treatment of collagenic eye disorder such as, for example, the treatment of keratoconus.

### FIELD OF THE INVENTION

The present invention relates to a novel treatment for collagenic eye disorders, especially disorders associated with collagen in the cornea and sclera, such as, for example, keratoconus. The present invention also relates to a pharmaceutical composition that is suitable for administration to the eye for the treatment of collagenic eye disorders.

### BACKGROUND OF THE INVENTION

Collagen-containing connective tissues play a fundamental role in maintaining the correct structure and function of the eye. The importance of collagen in the eye is demonstrated by its natural abundance, with approximately 80% of the eye comprising collagen. The properties of collagen, namely its strength and elasticity, help maintain the curved geometry of the eye ball, which is necessary for the eye to function properly.

In addition, collagen also helps the eye ball cope with changes in internal pressure by maintaining the necessary rigidity and elasticity that prevent the eye from bursting/rupturing and/or collapsing.

The weakening and/or degradation of structural proteins (such as collagen) in the eye is symptomatic of a number of eye disorders (referred to herein as collagenic eye disorders). Illustrative examples of collagenic eye disorders include various forms of corneal ectasia (non-inflammatory corneal ectasia, e.g. keratoconus, keratoglobus, pellucid marginal degeneration; inflammatory corneal ectasia; iatrogenic corneal ectasia (keratectasia), e.g. following laser refractive procedures/refractive surgery (LASIK, LASEK, PRK); or myopia). In addition, collagenic eye disorders include disorders in which the collagen in the eye, particularly the cornea or sclera, is weakened and/or degraded as a consequence of inflammation, infection, injury or corneal oedema.

By way of example, keratoconus is a degenerative disorder that results in a weakening of the collagen in the eye, which ultimately leads to progressive distortions in the shape of the eye ball. This progressive change in the eye's shape causes the eye to adopt a more conical shape over time which, in severe cases, can result in visual deterioration and eventual blindness.

Current methodologies for the treatment of keratoconus, and other related disorders, seek to strengthen the weakened collagen by photochemically cross-linking the collagen with riboflavin (vitamin B2). This technique, commonly known as corneal cross-linking or CXL. The procedure involves the application of riboflavin to the eye followed by exposure to UV radiation to initiate the photochemical cross-linking of the collagen with the riboflavin. However, the exposure of the eye to UV radiation can result in damage to the corneal endothelium and/or stromal cells. In the most severe cases, retinal degeneration can occur. The CXL procedure also requires the top layer of the cornea (epithelium) to be removed in order to enhance riboflavin penetration into the corneal stroma. The removal of the epithelium from the eye requires delicate surgical techniques and also carries a risk of infection and is painful for the patient. As a consequence, specially trained medical practitioners and equipment for the UV treatment are required in order to treat keratoconus and related disorders.

US2014/0271897 discloses a composition of 20 mg/ml disulfosuccinimidyl suberate in PBS pH 7.2. However, such compositions have stability issues. The pH of the composition within US2014/0271897 rapidly drops to a pH which is too low to be acceptable for application to the eye without significant discomfort to the patient and potentially damaging cells. Therefore, compositions which remain stable and retain the pH at an acceptable value are needed.

There is, therefore, a need for improved approaches for treating collagenic eye disorders in which cross-linking of collagen in the cornea and/or the sclera of the eye is beneficial.

The present invention was devised with the foregoing in mind.

### SUMMARY OF THE INVENTION

The present invention provides a novel treatment for collagenic eye disorders such as, for example, keratoconus.

Thus, according to a first aspect of the invention, there is provided a pharmaceutical composition suitable for administration to the eye, comprising:
(i) an aqueous vehicle;
(ii) a water soluble cross-linker dissolved in the aqueous vehicle, wherein the water soluble cross-linker is a compound of Formula (I) shown below, or a pharmaceutically acceptable salt and/or solvate thereof:

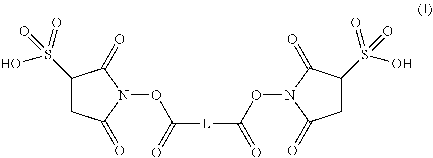

wherein L is a linear or branched (1-12C)alkylene linker that optionally comprises one or more heteroatoms selected from N, O or S, and is optionally substituted with one or more groups selected from carboxy, anhydride, oxo, halo, trifluoromethyl, cyano, nitro, hydroxy, mercapto, amino, (1-6C)alkyl, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, or (2-6C)alkanoyloxy;
   and wherein the composition further comprises a buffer to maintain the pH within a range of 6.0 to 8.5 for at least 15 minutes.

In another aspect, the present invention provides a pharmaceutical composition as defined herein for use in the treatment of a collagenic eye disorder.

In another aspect, the present invention provides a pharmaceutical composition as defined herein for use in the treatment of wounds (e.g. by stiffening tissue in or around a wound).

In another aspect, the present invention provides a method of treating a collagenic eye disorder, said method comprising administering to a human or animal subject in need of such treatment a therapeutically effective amount of a pharmaceutical composition as defined herein.

In yet another aspect, the present invention provides a device comprising a pharmaceutical composition as defined herein, wherein said device is configured to dispense a dose of the pharmaceutical composition to an eye of a patient.

In another aspect, there is provided a kit comprising
a. an aqueous vehicle;
b. a water soluble cross-linker as defined herein; and
c. either:
   a buffer comprised within the aqueous vehicle to maintain the pH within a range of 6.0 to 8.5 for at least 15 minutes after dissolving the water soluble cross-linker in the aqueous vehicle; or
   a buffer solution, wherein the buffer maintains the pH within a range of 6.0 to 8.5 for at least 15 minutes after mixing with the water soluble cross-linker and aqueous vehicle with the buffer solution.

### DETAILED DESCRIPTION OF THE INVENTION

### Definitions

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Unless otherwise stated, the following terms used in the specification and claims have the following meanings set out below.

The terms “treating” or “treatment” include prophylaxis as well as the alleviation of established symptoms of a disease or condition. “Treating” or “treatment” therefore includes: (1) preventing or delaying the appearance of clinical symptoms of the disease or condition developing in a subject that may be afflicted with or predisposed to the disease or condition, but does not yet experience or display clinical or subclinical symptoms of the disease or condition, (2) inhibiting the disease or condition, i.e., arresting, reducing or delaying the development of the disease or condition or a relapse thereof (in case of maintenance treatment) or at least one clinical or subclinical symptom thereof, or (3) relieving or attenuating the disease or condition, i.e., causing regression of the disease or condition or at least one of its clinical or subclinical symptoms.

Unless otherwise specified, where the quantity or concentration of a particular component of a given formulation is specified as a weight percentage (wt. % or % w/w), said weight percentage refers to the percentage of said component by weight relative to the total weight of the formulation as a whole. It will be understood by those skilled in the art that the sum of weight percentages of all components of a formulation will total 100 wt. %. However, where not all components are listed (e.g. where formulations are said to “comprise” one or more particular components), the weight percentage balance may optionally be made up to 100 wt % by unspecified ingredients (e.g. a diluent, such as water, or other non-essential but suitable additives).

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl.

The term “(m-nC)” or “(m-nC) group” used alone or as a prefix, refers to any group having m to n carbon atoms.

Unless otherwise stated, the term “collagenic eye disorder” refers to eye disorders that are associated with the weakening, degradation and/or damage to structural proteins, such as collagen, in the eye. Although it will be appreciated by a person skilled in the art that collagen is the main structural protein referred to herein, it will be understood that the term “collagenic eye disorder” also encompasses eye disorders associated with the weakening, degradation and/or damage of collagen in combination with other structural proteins in the eye. Furthermore, the term encompasses the weakening, degradation and/or damage to all parts of the eye, such as, for example, the cornea and the sclera.

### Compositions of the Present Invention

As previously stated, the present invention provides a pharmaceutical composition that is suitable for administration to the eye. Such compositions may be utilised in the treatment of collagenic eye disorders, such as, for example, keratoconus.

In a first aspect, the invention provides a pharmaceutical composition suitable for administration to the eye (ocular administration) comprising:
(i) an aqueous vehicle; and
(ii) a water soluble cross-linker dissolved in the aqueous vehicle, wherein the water soluble cross-linker is a compound of Formula (I) shown below, or a pharmaceutically acceptable salt and/or solvate thereof;

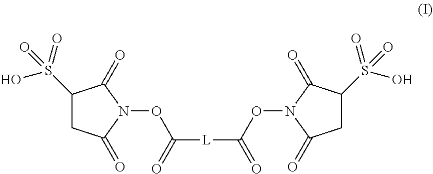

wherein L is a linear or branched (1-12C)alkylene linker that optionally comprises one or more heteroatoms selected from N, O or S, and is optionally substituted with one or more groups selected from carboxy, anhydride, oxo, halo, trifluoromethyl, cyano, nitro, hydroxy, mercapto, amino, (1-6C)alkyl, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, or (2-6C)alkanoyloxy;
and wherein the composition further comprises a buffer to maintain the pH within a range of 6.0 to 8.5 for at least 15 minutes.

### Cross-Linker

The cross-linkers of the present invention are stable in the pharmaceutical compositions defined herein and display excellent levels of collagen cross-linking. The cross-linkers of the present invention are also water soluble and are suitably non-toxic, making them particularly well suited for use in the treatment of collagenic eye disorders. Furthermore, when compared to the established corneal crosslinking (CXL) procedures, the cross-linkers of the present invention do not require any UV radiation in order to initiate the cross-linking and they can be administered to the eye without the need to remove the epithelium. The cross-linkers of the present invention are therefore viable alternative agents for the treatment of collagenic eye disorders, such as, for example, keratoconus.

The cross-linkers of the present invention water soluble. It will be understood by a person skilled in the art that water solubility is fundamental in allowing the cross-linker to effectively penetrate/permeate into the collagen tissue of the eye. Suitably, the cross-linkers of the present invention are also non-toxic and biocompatible.

Particular cross-linkers of the invention include, for example, cross-linkers of the formula (I), or pharmaceutically acceptable salts and/or solvates thereof, wherein, unless otherwise stated, L, and any associated substituent group has any of the meanings defined hereinbefore or in any of paragraphs (1) to (5) hereinafter:—
(1) L is a linear or branched (1-12C)alkylene, and is optionally substituted with one or more groups selected from carboxy, anhydride, oxo, halo, trifluoromethyl, cyano, nitro, hydroxy, mercapto, amino, (1-6C)alkyl, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, or (2-6C)alkanoyloxy;
(2) L is a (2-8C)alkylene, and is optionally substituted with one or more groups selected from carboxy, anhydride, oxo, halo, trifluoromethyl, cyano, nitro, hydroxy, mercapto, amino, (1-4C)alkyl, (1-4)alkoxy, (1-4)alkylthio, (1-4C)alkylsulphinyl or (1-4C)alkylsulphonyl.
(3) L is a (5-7C)alkylene and is optionally substituted with one or more groups selected from carboxy, anhydride, oxo, halo, trifluoromethyl, cyano, nitro, hydroxy, mercapto and amino.
(4) L is a (5-7C)alkylene.
(5) L is a (6C)alkylene

In an embodiment of the compounds of formula (I), L is as defined in any one of paragraphs (1) to (5) above. In a further embodiment, L is defined as in any one of paragraphs (3) to (5) above. In yet another embodiment, L is as defined in paragraph (4) above.

In a preferred embodiment, L is (6C)alkylene, e.g. the water soluble cross-linker is bissulfosuccinimidyl suberate, or a pharmaceutically acceptable salt and/or solvate thereof and optionally the disodium salt.

The cross-linker of the present invention may be present at any suitable concentration. In an embodiment, the concentration of the cross-linker in the pharmaceutical composition of the present invention is from 0.01 M to 0.3 M. Suitably, the concentration of the cross-linker in the pharmaceutical composition of the present invention is from 0.1 M to 0.25 M. More suitably, the concentration of the cross-linker in the pharmaceutical composition of the present invention is from 0.15 M to 0.25 M.

A suitable pharmaceutically-acceptable salt of a cross-linker of the invention is, for example, an alkali or alkaline earth metal salt such as a sodium, calcium or magnesium salt, or an ammonium salt, or a salt with an organic base such as methylamine, dimethylamine, trimethylamine, piperidine, morpholine or tris-(2-hydroxyethyl)amine. A further suitable pharmaceutically-acceptable salt of a cross-linker of the invention is, for example, a salt formed within the human or animal body after administration of a cross-linker of the invention.

A suitable pharmaceutically-acceptable solvate of a cross-linker of the invention is, for example, a hydrate such as a hemi-hydrate, a mono-hydrate, a di-hydrate or a tri-hydrate or an alternative quantity thereof.

### Aqueous Vehicle

The water soluble cross-linkers of the present invention are dissolved, either fully or partially, in an aqueous vehicle. The term ‘aqueous vehicle’ can be understood to mean a liquid vehicle which predominately contains water.

The aqueous vehicle may therefore comprise greater than about 50% by volume of water. For example, the aqueous medium may contain more than 60% by volume water, e.g. more than 75% by volume water or more than 95% by volume water. Typically, the aqueous vehicle will comprise between 75 to 100% by volume of water.

The ‘aqueous vehicle’ may also comprise other solvents. It may therefore comprise organic solvents which may be fully or partially miscible with water. The aqueous medium may comprise solvents which are miscible with water, for example alcohols (e.g. methanol and ethanol). The aqueous medium may also comprise additives which may be ionic, organic or amphiphilic. Examples of such additives include surfactants, viscosity modifiers, tonicity agents, sterilising agents and a solubility enhancers.

Non-limiting examples of suitable surfactants include stearates, glycerides and cyclodextrins.

### Buffer

The pharmaceutical composition of the present invention comprises a buffer in order to maintain the composition at a pH compatible for use in the eye.

In an embodiment, the buffer maintains the pH of the composition within a range of 6.0 to 8.0. In a further embodiment, the buffer maintains the pH of the composition is within the range 6.5 to 8.0.

In an embodiment, the buffer maintains the pH of the composition within the stated range for at least 30 minutes, at least 1 hour, at least 2 hours, at least 6 hours, at least 24 hours or at least 48 hours. Preferably, the buffer maintains the pH of the composition within the stated range for at least 24 hours or at least 48 hours.

Suitably, the buffer is present in a concentration of at least 0.05M, suitably at least 0.1M, more suitably at least 0.5M. Suitably, the buffer is present in a concentration of from 0.05M to 2M, e.g. from 0.1 to 1M.

It will be understood that any suitable buffer may be used. In an embodiment, the buffer is selected from the group comprising: phosphate, acetate, citrate, sulfonic acid, ascorbate, linolenate or carbonate-bicarbonate based buffers. In a further embodiment, the buffer is selected from the group comprising: phosphate, acetate, citrate, sulfonic acid or carbonate-bicarbonate based buffers. Suitably, the buffer is selected from a phosphate buffer or a carbonate-bicarbonate based buffer.

In a particular embodiment, the buffer is a carbonate-bicarbonate buffer (e.g. a sodium carbonate and sodium bicarbonate buffer).

Suitably, the buffer is a carbonate-bicarbonate buffer and the molar ratio of carbonate to bicarbonate is from 0.05:1 to 20:1 (e.g. from 0.5:1 to 20:1) more suitably from 0.1:1 to 15:1 (e.g. from 0.75:1 to 15:1), most suitably from 1:1 to 10:1.

The composition may further comprise an additional buffer, for example phosphate buffered saline (PBS).

### Additional Excipients

It will be appreciated that the pharmaceutical compositions of the present invention may comprise additional pharmaceutical excipients. Additional excipients may be included to improve various properties of the formulation, such as, for example, formulation stability, biocompatibility and administrability. A person skilled in the art will be able to select suitable excipients to include based on conventional knowledge in the formulation field.

A non-limiting list of possible additional excipients that may be added to the pharmaceutical compositions of the present invention include: pH modifiers, surfactants, viscosity modifiers, tonicity agents, sterilising agents, preservatives, lubricants and solubility enhancers.

In an embodiment, the pharmaceutical compositions may also comprise one or more additional therapeutic agents, such as, for example, antibiotics, steroids, anaesthetics and/or antihistamines.

### Therapeutic Uses and Applications

### Pharmaceutical Compositions

The present invention provides a pharmaceutical composition as defined herein for use as a medicament.

The pharmaceutical compositions of the present invention are particularly suited to the treatment of collagenic eye disorders. Once administered, the pharmaceutical compositions deliver the cross-linker to the eye, thereby initiating the crosslinking of collagen within the eye and restoring structural integrity to the eye.

Collagenic eye disorders that can be treated with the pharmaceutical compositions defined herein include:
1. corneal ectasia, including:
   (i) non-inflammatory corneal ectasia—e.g. keratoconus, keratoglobus, pellucid marginal degeneration;
   (ii) inflammatory corneal ectasia;
   (iii) iatrogenic corneal ectasia (keratectasia)—e.g. following laser refractive procedures/refractive surgery (LASIK, LASEK, PRK)];
   (iv) myopia (i.e. crosslinking of collagen in sclera to treat progressive myopia);
2. inflammation in the eye*, including:
   (i) treatment of the cornea resulting from infective, traumatic (chemical, physical, thermal, surgical) or immune-mediated** (including vasculitic)** corneal disease;
   (ii) sclera resulting from infective, traumatic (chemical, physical, thermal, surgical) or immune-mediated** (including vasculitic)** scleral disease, including scleromalacia perforans;
3. re-shaping of the eye, optionally following surgery; for example reshaping of the cornea, including:
   (i) reshaping the donor cornea for transplantation***
   (ii) stabilising corneal shape before or after refractive surgical procedures;
4. corneal swelling due corneal oedema (e.g. bullous keratopathy, Fuchs Endothelial Dystrophy, Congenital Hereditary Endothelial Dystrophy, hydrops of the cornea in keratoconus)
   * by reducing/inhibiting inflammation by destroying inflammatory cells and making the tissue more resistant to digestion; for example, stopping inflammatory mediated cellular damage and enzymatic destruction of collagen or proteoglycans and treatment of corneal or scleral stromal ulceration and melts.
   ** as a result of connective tissue disease, such as rheumatoid arthritis (RA), Sjögren syndrome, Mooren ulcer, or any systemic vasculitic disorder/collagen vascular disease (eg, systemic lupus erythematosus [SLE], Wegener granulomatosis, polyarteritis nodosa).

The pharmaceutical compositions defined herein may also be used in mechanically strengthening a weakened cornea or sclera in the treatment or prevention of:
(i) myopia—crosslinking of collagen in sclera to treat progressive myopia;
(ii) glaucoma—scleral cross-linking to alter biomechanical properties of the optic nerve head and lamina cribrosa
(iii) microbial keratitis
(iv) progressive diseases that cause change in size or thickness of the eye ball or its part (e.g. megalocornea, buphthalmos, peripheral ulcerative keratitis)
(v) complications arising from refractive surgical procedures that weaken the cornea and/or sclera.
   *** for better tissue healing and refractive outcomes.

Thus, the present invention provides a pharmaceutical composition as defined herein for use in the treatment of a collagenic eye disorder.

Suitably, the collagenic eye disorder is a disorder associated with the weakening, degradation and/or damage to collagen in the cornea and/or sclera of the eye.

Suitably, the pharmaceutical composition as defined herein is
(1) for use in the treatment of:
   i. corneal ectasia, including: (i) non-inflammatory corneal ectasia—e.g. keratoconus, keratoglobus, pellucid marginal degeneration; (ii) inflammatory corneal ectasia; (iii) iatrogenic corneal ectasia (keratectasia); (iv) myopia;
   ii. inflammation in the eye caused by infective, traumatic (chemical, physical, thermal, surgical) or immune-mediated (including vasculitic) corneal or scleral disease;
   iii. re-shaping the eye, optionally following transplant or surgery; or
   iv. corneal swelling due corneal oedema (e.g. bullous keratopathy, Fuchs Endothelial Dystrophy, Congenital Hereditary Endothelial Dystrophy, hydrops of the cornea in keratoconus);
      or
(2) for use in mechanically strengthening a weakened cornea and/or sclera in the treatment or prevention of:
   i. myopia
   ii. glaucoma
   iii. microbial keratitis
   iv. progressive diseases that cause change in size or thickness of the eye ball or its part (e.g. megalocornea, buphthalmos, peripheral ulcerative keratitis)
   v. complications arising from refractive surgical procedures that weaken the cornea and/or sclera.

Suitably, the pharmaceutical composition as defined herein is for use in the treatment of keratoconus.

In another embodiment, the pharmaceutical composition as defined herein is for use in stiffening the cornea in corneal transplant procedures to make it more robust for suturing and/or to reduce the corneal astigmatism.

In another embodiment, the pharmaceutical composition as defined herein is for use as a treatment in wound care to stiffen the tissue around a wound, for example to make it more robust for suturing.

In another aspect, the present invention provides a method of treating a collagenic eye disorder, said method comprising administering to a human or animal subject in need of such treatment a therapeutically effective amount of a pharmaceutical composition as defined herein.

Suitably, the collagenic eye disorder is a disorder associated with the weakening, degradation and/or damage to collagen in the cornea and/or sclera of the eye.

Suitably, either:
(1) the collagenic eye disorder to be treated is selected from:
   i. corneal ectasia, including: (i) non-inflammatory corneal ectasia—e.g. keratoconus, keratoglobus, pellucid marginal degeneration; (ii) inflammatory corneal ectasia; (iii) iatrogenic corneal ectasia (keratectasia); (iv) myopia;
   ii. inflammation in the eye caused by infective, traumatic (chemical, physical, thermal, surgical) or immune-mediated (including vasculitic) corneal or scleral disease;
   iii. re-shaping the eye, optionally following transplant or surgery; or
   iv. corneal swelling due corneal oedema (e.g. bullous keratopathy, Fuchs Endothelial Dystrophy, Congenital Hereditary Endothelial Dystrophy, hydrops of the cornea in keratoconus); or
(2) the method involves mechanically strengthening a weakened cornea and/or sclera in the treatment or prevention of:
   i. myopia
   ii. glaucoma
   iii. microbial keratitis
   iv. progressive diseases that cause change in size or thickness of the eyeball or its part (e.g. megalocornea, buphthalmos, peripheral ulcerative keratitis)
   v. complications arising from refractive surgical procedures that weaken the cornea and/or sclera.

Suitably, the collagenic eye disorder to be treated is keratoconus.

The invention also provides a method of corneal transplantation, wherein the method comprises treating the host and donor cornea of the eye in a patient in need of such treatment with a pharmaceutical composition as defined herein to stiffen the cornea to make it more robust for suturing.

The cross-linker and pharmaceutical compositions defined herein may be used to treat and collagenic eye disorder. Collagenic eye disorders are any eye disorder or medical application that is associated with the weakening, degradation and/or damage to collagen in the eye. The term encompasses the weakening, degradation and/or damage to all parts of the eye, such as, for example, the cornea and the sclera.

The crosslinker and pharmaceutical compositions defined herein may also be used to aid wound healing. The crosslinker and pharmaceutical compositions may be used to stiffen tissue around a wound to aid suturing.

The pharmaceutical compositions of the present invention may be used on their own as the sole therapy. Alternatively, the compositions may be administered as part of a combination therapy with one or more other eye treatments. Such conjoint treatment may be achieved by way of the simultaneous, sequential or separate administration of the individual components of the treatment.

By way of example, collagenic eye disorders may result in a number of other undesirable symptoms to the patient, such as, for example, pain, infection, dryness and discomfort. Accordingly, the pharmaceutical compositions of the present invention may be used in combination with one or more additional medicaments or additives, such as, for example, hydrating agents, antibiotics, steroids, anaesthetics and antihistamines.

The pharmaceutical compositions of the present invention may be presented as topical formulations for administration to the eye, e.g. eye drops (including viscous eye drops), eye sprays, eye washes or eye creams/ointments.

The pharmaceutical compositions of the present invention may be presented as topical formulations for administration to a wound, e.g. creams/ointments.

### Administration Devices

The pharmaceutical compositions of the present invention may be incorporated into a suitable device to deliver a dose of the composition to the eye during use.

Thus, there is provided a device comprising a pharmaceutical composition as defined herein, wherein said device is configured to dispense a dose of the pharmaceutical composition to an eye of a patient

Any suitable device known in the art may be used, such as, for example, a conventional eye drop bottle.

### DETAILED DESCRIPTION OF THE DRAWINGS

The present invention is further defined with reference to the accompanying figures which are described below:

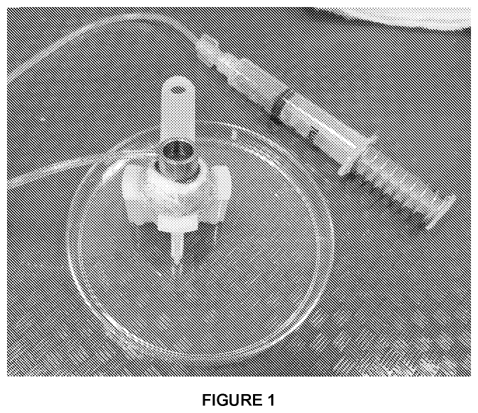

FIG. 1 shows an E. Janach suction ring attached to ex vivo porcine cornea.

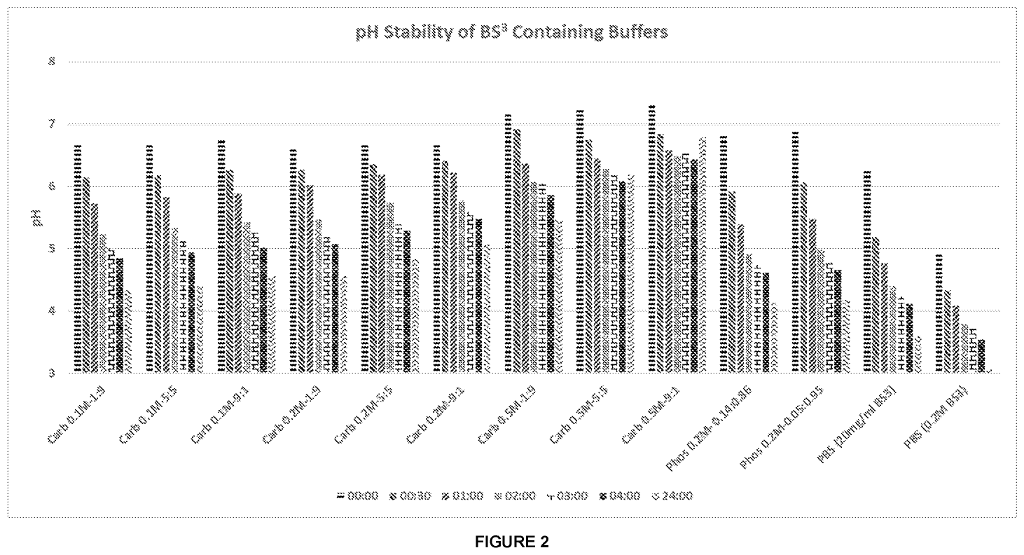

FIG. 2 shows graphs detailing the pH stability of different BS3 containing buffering systems.

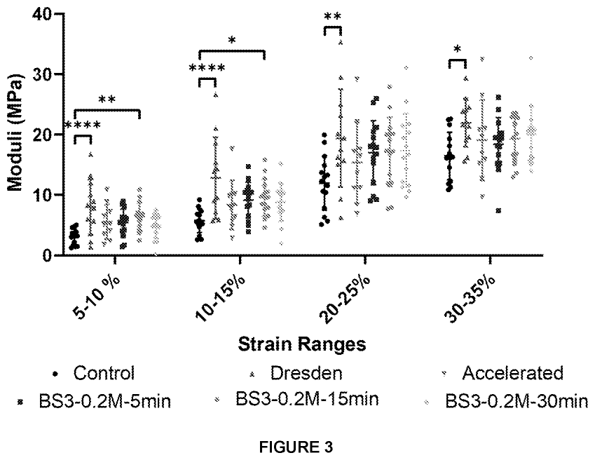

FIG. 3 shows scatter plot (displaying mean and standard deviation) of elastic moduli of control corneas, corneas treated with Dresden or Accelerated UV/Riboflavin protocols and BS³ treated samples at a strain ranges of 5-10%, 10-15%, 20-25% and 30-35%. BS³ treatment times of 5, 15 and 30 minutes were measured.

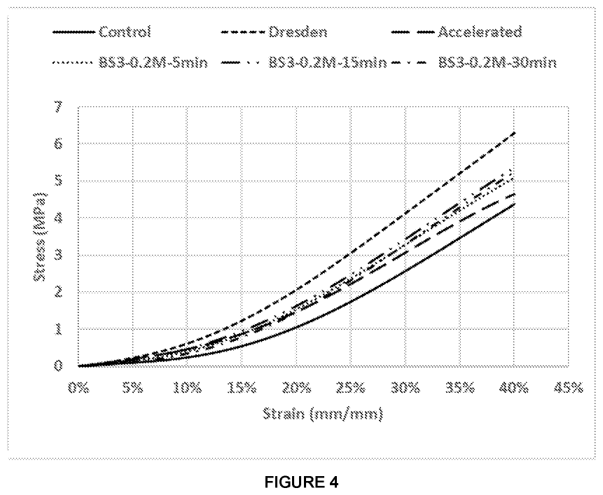

FIG. 4 shows a line graph displaying averaged stress-strain behaviour or corneas subjected to uniaxial tensile testing.

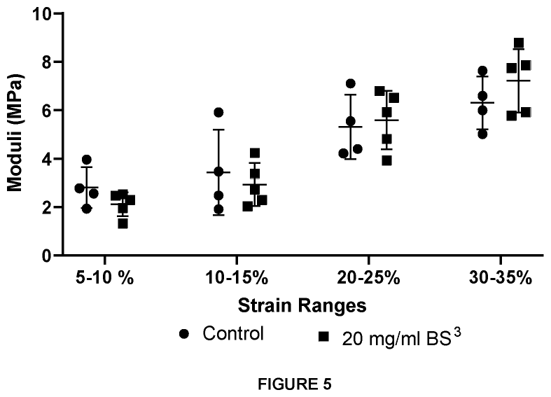

FIG. 5 shows a scatter plot (displaying mean and standard deviation) of elastic modulus of control and BS³ treated samples using formulation mentioned in US2014/0271897 A1 paragraph [0358] at in strain ranges of 5-10%, 10-15%, 20-25% and 30-35%.

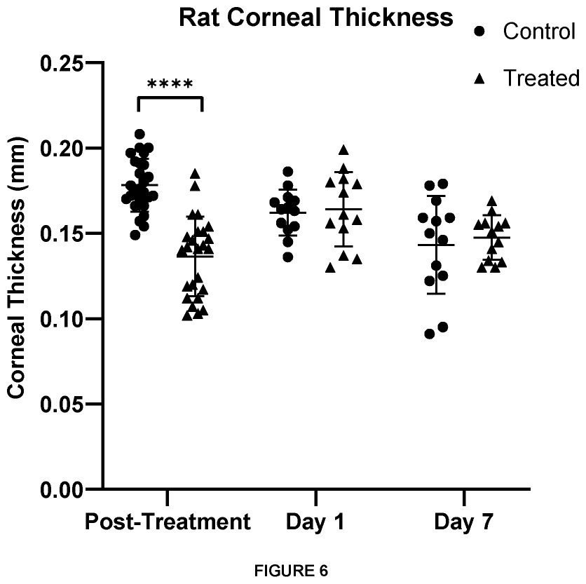

FIG. 6 shows a scatter plot (displaying mean and standard deviation) of rat corneal thickness of treated and untreated contralateral control eyes measured with OCT, immediately after treatment (Post-Treatment), day 1 and day 7 after treatment with BS³. Treated eyes were compared with contralateral control eyes at each time point by paired t-test. Treated eyes had a significant reduction in thickness immediately after treatment (p=<0.0001) which resolved by day 1. Post-treatment n=25, Day 1 n=13 and Day 7 n=13.

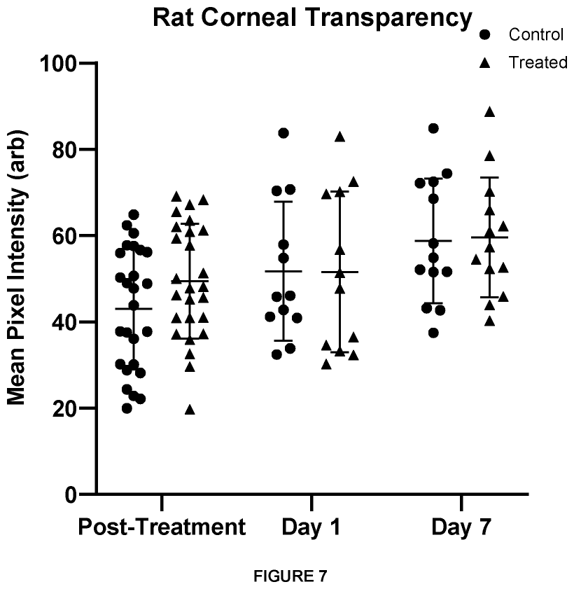

FIG. 7 shows a scatter plot (displaying mean and standard deviation) of rat corneal transparency of treated and untreated contralateral control eyes measured with OCT, immediately after treatment (Post-Treatment), day 1 and day 7 after treatment with BS³. Data represent mean intensity of cornea pixels minus background intensity determined from OCT images. Treated eyes were compared with contralateral control eyes at each time point by paired t-test. No significant differences were observed at any time point (p>0.05). Post-treatment n=25, Day 1 n=13 and Day 7 n=13.

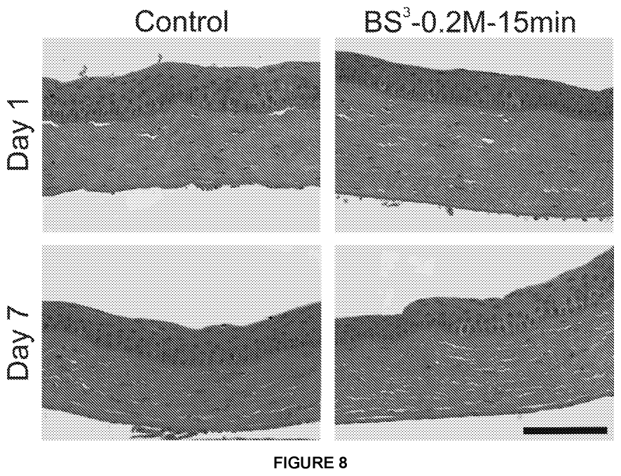

FIG. 8 shows a H&E stained corneal sections from rat eyes, control and BS³ treated, at 1- and 7-days post-treatment. H&E did not reveal any structural changes between treated and contralateral untreated control eyes. Scale bar=500 μm.

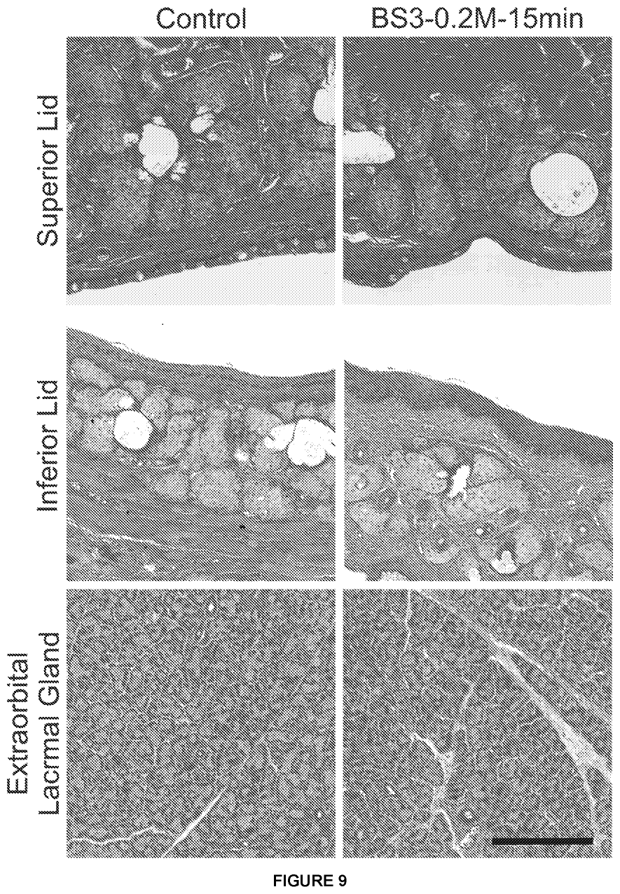

FIG. 9 shows H&E stained sections of rat superior and inferior lids, and extraorbital lacrimal glands, control and BS³ treated. Superior lids and extraorbital lacrimal glands were collected 24 hours post-treatment. Inferior lids are 7 days post-treatment due to processing issues with earlier samples. No structural changes or signs of toxicity were observed.

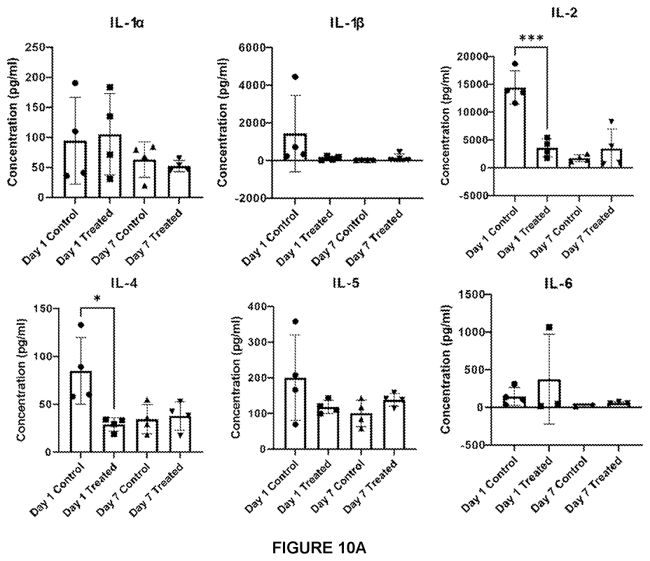

FIGS. 10A and 10B show Inflammatory cytokine changes in rat aqueous humour. Data presented as mean±standard deviation. *=p<0.5, ***=P<0.001.

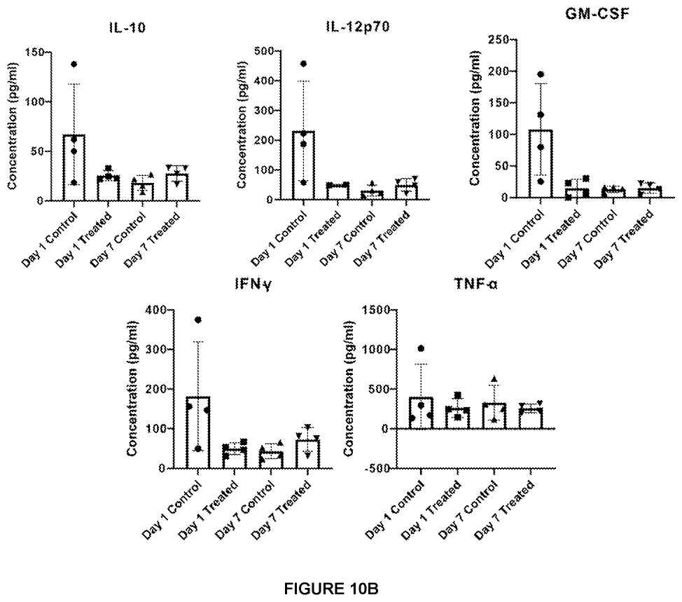

FIG. 11 shows a scatter plot (displaying mean and standard deviation) of elastic modulus of rabbit corneas 3 days after treatment with 0.2M BS³ for 15 mins in comparison with the contralateral control untreated eye, n=14. No significant difference at lower strain ranges but the was a significant 43% increase in stiffness at the 15-20% strain range and a 104% increase at the 20-25% strain range.

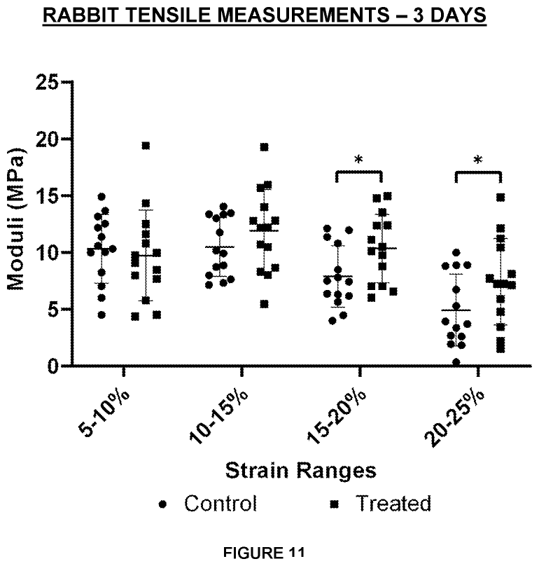

FIG. 12 shows a scatter plot (displaying mean and standard deviation) of elastic modulus of rabbit corneas 28 days after treatment with 0.2M BS³ for 15 mins in comparison with the contralateral control untreated eye, n=14. No significant difference at lowest and highest strain ranges but there was a significant 29% increase in stiffness at the 10-15% strain range and a 28% increase at the 15-20% strain range.

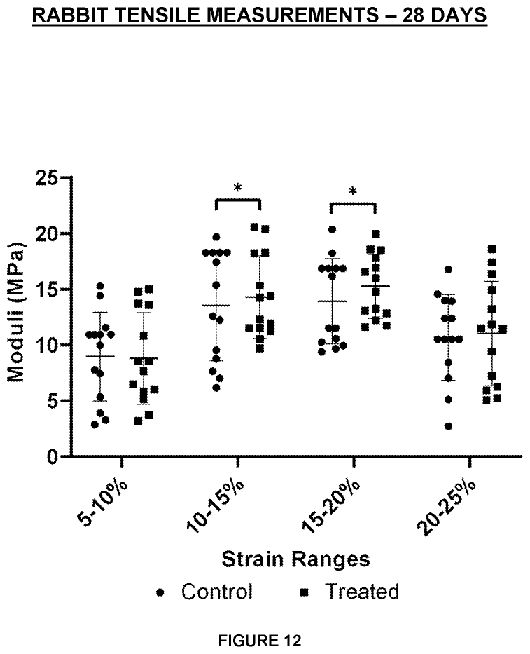

FIG. 13 shows Scatter plot (displaying mean and standard deviation) of mixed sex rabbit corneal transparency of treated and untreated contralateral control eyes measured with OCT, immediately after treatment (Post-Treatment) and 3 days after treatment with BS³. Data represent mean intensity of cornea pixels minus background intensity determined from OCT images. Treated eyes were compared with contralateral control eyes at each time point by paired t-test, n=11. Treated eyes had a significant increase in opacity, indicated by increased pixel intensity, immediately after treatment (p=<0.0001); this was resolved by day 3 (p=0.3309).

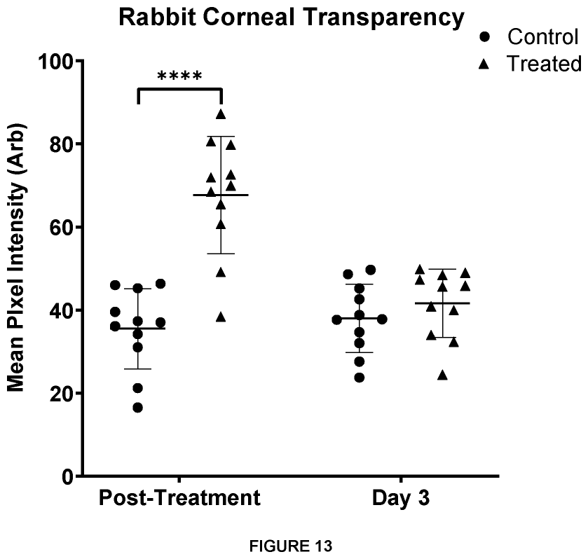

FIG. 14 shows H&E stained micrographs of rabbit cornea 3 days after treatment with 0.2M BS³ for 15 mins and contralateral controls demonstrating no difference between the untreated (A) and treated (B) cornea with the presence of a stratified epithelium and a structurally similar stroma. Comparisons of higher magnification untreated (C) and treated (D) epithelium also display similar multi-layered structures. An endothelium was also present on control (E) and treated (F) corneas. The control endothelium has separated from the Descemet's Membrane, but this is a processing artefact. A-B scale bar 250 μm, C-F scale bar=50 μm.

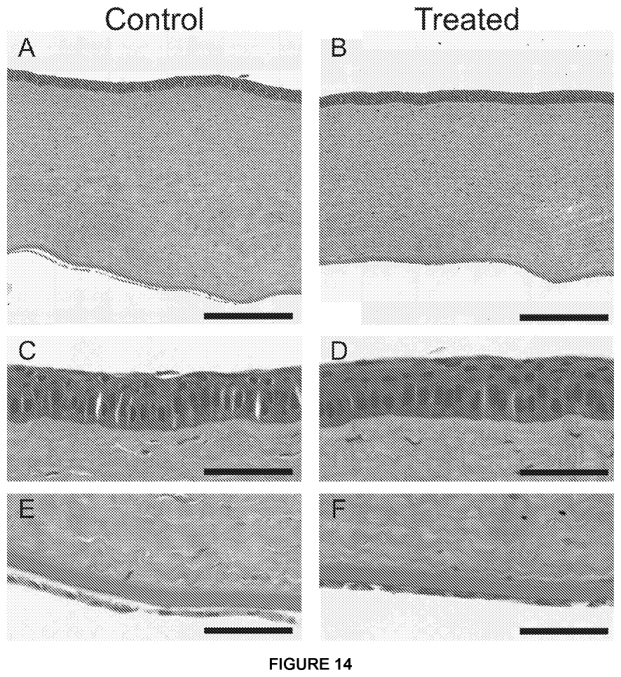

FIG. 15 shows a scatter plot (displaying mean and standard deviation) of mixed sex rabbit corneal thickness of treated and untreated contralateral control eyes measured with OCT, immediately after treatment (Post-Treatment) and 3 days after treatment with BS³. Treated eyes were compared with contralateral control eyes at each time point by paired t-test, n=11. Treated eyes had a significant reduction in thickness immediately after treatment (p=<0.0001). There was still a significant reduction in thickness at day 3 (p=0.0332), however this was only 11 μm.

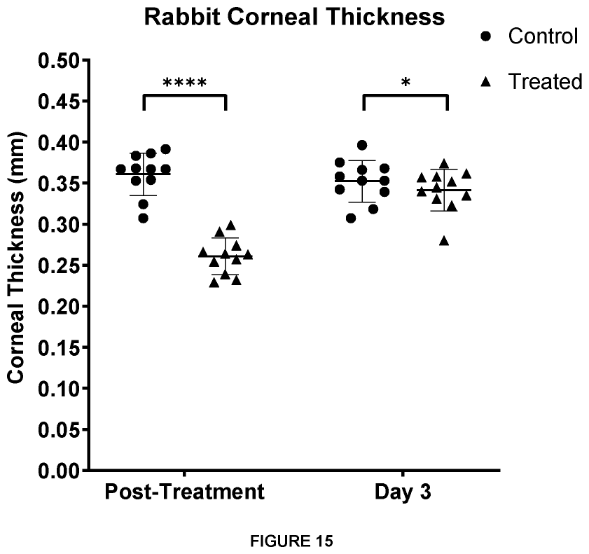

FIG. 16 shows a bar chart (displaying mean and standard deviation) of mixed sex rabbit corneal thickness of treated and untreated contralateral control eyes measured with OCT, immediately after treatment (Post-Treatment) and 28 days after treatment with BS³.

While specific embodiments of the invention have been described herein for the purpose of reference and illustration, various modifications will be apparent to a person skilled in the art without departing from the scope of the invention as defined by the appended claims.

### EXAMPLES

### Materials and Methods

### Materials

### Buffer Preparation

To investigate cross-linker stability 0.2 M BS³ was prepared in various buffers. The pH was measured using a SevenGO SG2 pH meter with an InLab Ultra-Micro-ISM electrode (Mettler Toldedo, Leicester, UK). Nine carbonate buffer and two phosphate buffer formulations were investigated. All were prepared in deionised H₂O. Formulations can be seen in Table 1 below. The pH of BS³ at concentrations of 0.2 M and 0.035 M (20 mg/ml as per patent US2014/0271897 A1 claim [0358]) in phosphate buffered saline (PBS) were also measured. The pH was measured immediately after addition BS3 and filtration (00:00) then at 30 mins, 1 hour, 2 hours, 3 hours, 4 hours, 24 hours.

### BS3 Preparation

BS³ (bis(sulfosuccinimidyl)suberate, Thermo Scientific Pierce, IL, USA) solutions was prepared to concentrations of 0.2 M in the buffer solution, vortexed for 1 min and then filtered through 0.2 μm filter.

### Ex Vivo Studies

### Porcine Tissue Collection

Eyes of the domestic pig (Sus domesticus) are collected from a local abattoir 0-4 hours post-mortem. Eyes are cleaned of excess tissue and fat and washed in PBS containing 200 U penicillin and 0.2 mg/ml streptomycin and 5 μg/ml amphotericin B (Merch Life Science, Dorset, UK). The eyes are stored in a petri dish containing the PBS antibiotic/antimycotic solution, corneas facing up, at 4° C. overnight.

### BS3 Cross-Linking of Porcine Corneas

The porcine eyes are removed from 4° C. and allowed to warm to room temperature. One-to-two drops of Minims Proxymetacaine hydrochloride 0.5% w/v (Bosch & Lomb, Surrey, UK) were applied to the cornea for 15 minutes, with additional drops added ever 5 minutes and then 2-3 minutes before treatment. After which a suction ring (E.Janach, Como, Italy) was attached to the cornea (FIG. 1) and 200 μl of BS³ solution was added inside the ring. Eyes were incubated for 5, 15 or 30 mins at 37° C.; after which the BS³ solution was aspirated and retained for pH measurement, and eyes were rinsed in PBS then stored, cornea facing down, in a 6-well plate with fresh PBS until tensile measurement.

TABLE 1
BS3 buffer formulations.

| | | | Ratio of | |
|---|---|---|---|---|
| Component | Component | Molarity of | Components | |
| 1 | 2 | Components | (C1:C2) | Abbreviation |
| Sodium | Sodium | 0.1M | 1:9 | Carb |
| Carbonate | Bicarbonate | | | 0.1M-1:9 |
| decahydrate | | | | |
| Sodium | Sodium | 0.1M | 5:5 | Carb |
| Carbonate | Bicarbonate | | | 0.1M-5:5 |
| decahydrate | | | | |
| Sodium | Sodium | 0.1M | 9:1 | Carb |
| Carbonate | Bicarbonate | | | 0.1M-9:1 |
| decahydrate | | | | |
| Sodium | Sodium | 0.2M | 1:9 | Carb |
| Carbonate | Bicarbonate | | | 0.2M-1:9 |
| decahydrate | | | | |
| Sodium | Sodium | 0.2M | 5:5 | Carb |
| Carbonate | Bicarbonate | | | 0.2M-5:5 |
| decahydrate | | | | |
| Sodium | Sodium | 0.2M | 9:1 | Carb |
| Carbonate | Bicarbonate | | | 0.2M-9:1 |
| decahydrate | | | | |
| Sodium | Sodium | 0.5M | 1:9 | Carb |
| Carbonate | Bicarbonate | | | 0.5M-1:9 |
| decahydrate | | | | |
| Sodium | Sodium | 0.5M | 5:5 | Carb |
| Carbonate | Bicarbonate | | | 0.5M-5:5 |
| decahydrate | | | | |
| Sodium | Sodium | 0.5M | 9:1 | Carb |
| Carbonate | Bicarbonate | | | 0.5M-9:1 |
| decahydrate | | | | |
| Potassium | Sodium | 0.2M | 0.14:0.86 | Phos 0.2M- |
| Phosphate | phosphate | | | 0.14:0.86 |
| Monobasic | dibasic | | | |
| Anhydrous | dodecahydrate | | | |
| Potassium | Sodium | 0.2M | 0.05:0.95 | Phos 0.2M- |
| Phosphate | phosphate | | | 0.05:0.95 |
| Monobasic | dibasic | | | |
| Anhydrous | dodecahydrate | | | |

### UV/Riboflavin Cross-Linking

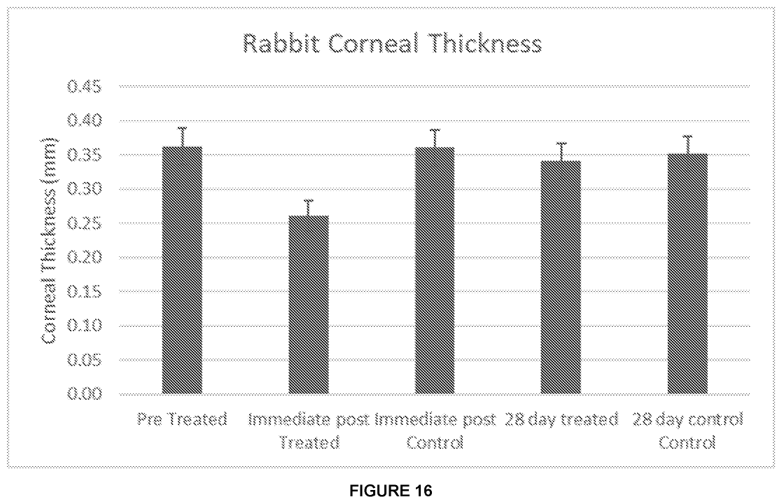

Suction rings were applied to porcine corneas as in FIG. 1 and 18% ethanol solution was applied for 30 seconds before being adsorbed by a sponge spear. The cornea was then rinsed with PBS. After this the epithelium was removed with a corneal scraper and sponge spear. Once the epithelium was removed 0.1% riboflavin (Vibex Rapid™, Avedro Inc, MA, USA) was applied every 3 mins. Riboflavin was applied for a total of 30 mins for the Dresden protocol and 15 minutes for the Accelerated protocol.

After riboflavin treatment eyes were cross-linked by UVA light using and KXL System (Avedro Inc). UV light was applied at 3 mW/cm² for 30 mins for the Dresden protocol and 6 mW/cm² for 15 mins for the Accelerated protocol for a total applied energy of 5.4 J/cm² for both protocols.

### Tensile Testing

The anterior section of the eye was removed by cutting around the globe 5-10 mm posteriorly from the limbus. The iris and ciliary process were removed from the posterior section of the tissue leaving only sclera and cornea. A strip of tissue was cut using a custom-made replaceable razor blade jig. Tissue thickness and width (determined as the distance between cutting edges) were measured with calipers.

The tissue strip was mounted in the grips of a Univert tensile tester (CellScale, Ontario, Canada) with a 50 N load cell. Tissue was stretched at a rate of 125 μm/s until failure. Data was captured at 1 Hz. Stress-strain curves were plotted and the elastic modulus was determined at a range of strains: 5-10%, 10-15%, 20-25% and 30-35%. Data were trimmed to 3% from the strain at ultimate tensile strain and 6 term polynomial equations we determined from each curve. Each equation had an R²>0.99. These polynomials represent the stress-stain response of each cornea and were used to create an average stress-strain curve for the samples.

### Statistical Analysis

Statistical analyses were performed with Prism 9.3.1 (GraphPad Software, San Diego, CA, USA). Ex vivo mechanical data were compared by One-way ANOVA and Tukey's post-hoc.

### In Vivo Studies

### Treatment of Animals

Male Wistar rats (300 g) and mixed sex New Zealand rabbits (1.5-3 kg), both specific-antigen free, were ordered from Charles River UK and ENVIGO, respectively. They were allowed a minimum of 7 days acclimatisation prior to procedures. Animals were anaesthetised and proxymetacaine hydrochloride 0.5% w/v was applied to the corneas. For the rats, a 2 cm section of silicone tubing was placed on the cornea whereas for the rabbits the suction ring (as above) was placed on the cornea, and 200 μl of the sterile filtered 0.2 M BS³ solution was instilled for 15 mins. Following this, eyes were rinsed with saline. Animals were provided with antibiotic/steroid drops (Tobradex, Novartis Pharmaceuticals UK Ltd, London, UK) daily and were monitored for any signs for distress or ocular discomfort.

The animals were euthanised at pre-determined time points (rats at 24 hours and 7 days; rabbits at 3 days post-treatment). Aqueous humour was collected. Rat ocular tissue was collected and fixed in 10% paraformaldehyde for histology. Rabbit the eyes enucleated and prepared for mechanical testing which was conducted the following day and the remaining cornea was fixed. Ocular tissue was also fixed in 10% paraformaldehyde.

### Corneal Thickness and Transparency

Corneal thickness and transparency were determined using optical coherence tomography (OCT) (ENVISU R2210, Leica Microsystems, Milton Keynes, UK) before treatment and prior to euthanasia. Corneal transparency was determined by the intensity of the pixels within the cornea minus background intensity from the corneal sections of the OCT images.

### Histology

Ocular tissue from the rats and that remaining after collection of samples for mechanical testing from the rabbits were processed in embedded in paraffin for histology.

### Aqueous Humour Cytokine Multiplex

Rat aqueous humour samples were collected immediately after culling using a 25G 0.5 U insulin syringe and needle. Samples were stored at −80° C. until testing with a Bio-Plex Pro™ Rat Cytokine Th1/Th2 Assay (Bio-Rad Laboratories Ltd., Warford, UK) following the manufacturer's instructions. Samples were measured using a Bio-plex 200 system with Bio-Plex Manager 5.0 software (Bio-Rad Laboratories Ltd., Warford, UK). Depending on volumes collected and sample required between aqueous from between 2-4 eyes were polled and were diluted by a factor of between 2-4.5. This created n=4 pools of each parameter, 3 technical replicates were performed.

### Tensile Testing

Rabbit eyes were collected immediately after culling and were stored in a petri dish containing the PBS antibiotic/antimycotic solution, corneas facing up, at 4° C. overnight. Mechanical testing was conducted using the protocol above for ex vivo porcine tissue.

### Statistical Analysis

Statistical analyses were performed with Prism 9.3.1 (GraphPad Software, San Diego, CA, USA). In vivo data were compared by paired t-tests to contralateral control eyes.

### Results

### Buffer Stability

The sodium carbonate: sodium bicarbonate-based buffers provided greater stability than the potassium phosphate: sodium phosphate buffers (FIG. 2). Increasing the molarity of the carbonates to 0.5 M increased the initial pH, after the addition of 0.2 M BS³, to >7 and provided the greatest stability. Precipitation was observed in 0.1 M and 0.2 M carbonate buffers at 24 hours. Increasing the carbonate:bicarbonate ratio of the 0.5 M buffers also increased starting pH and stability; for carbonate:bicarbonate ratios of 5:5 and 9:1 had pH>6 over the 24 hour testing period and did not have any precipitation. The phosphate buffers exhibited poor stability. Standard laboratory PBS (Dulbecco A) provided the least stable formulation, even with the reduced BS³ concentration stated in patent US2014/0271897 A1 claim [0358] of 20 mg/ml (0.035 M) the pH had dropped to 5.18 by 30 minutes. The 0.5 M 9:1 Carbonate:Bicarbonate buffer was the most stable over the time and thus was chosen for subsequent testing.

### Ex Vivo

### Ex Vivo Tensile Testing

In our study corneas were treated with 0.2 M BS³ in 0.5 M sodium carbonate:bicarbonate buffer 9:1 ratio for 5, 15 and 30 minutes. At a strain range of 5-10% these treatment times achieved stiffnesses of 5.43±2.23, 6.47±2.23, and 4.84±2.02 MPa respectively, whereas the control corneas had an average stiffness of 3.1±1.25 MPa (FIG. 3). Of the BS³ treatments only the 15 min treatment was significantly different from control (p=0.0034) with a 109% increase in stiffness (Table 2). Corneas treated with Dresden and Accelerated UV/Riboflavin protocols were also measured and had moduli of 7.99±4.58 and 5.45±2.97 MPa, respectively, at a strain range of 5-10%. The Dresden protocol produced a modulus significantly different to control (p<0.0001) with a 158% increase in stiffness. The Accelerated protocol was not significantly different.

At a strain range of 10-15% the Dresden UV/riboflavin corneas maintained a significant (p<0.0001) increase in stiffness (123%) compared to control corneas. The BS³-0.2M-15 min corneas were also still significantly different to controls in the 10-15% strain range (p=0.0275, 68% increase). At strain ranges of 20-25% and 30-25% only the Dresden UV/riboflavin treatment produced significant differences to controls (p=0.0099, 62% and p=0.0111, 34%) respectively.

The averaged stress-strain responses of the various parameters can be seen in FIG. 4. The greatest divergence in stress-strain behaviour occurs at lower strains, between ˜5-15%. This is reflected in the analysis of the elastic moduli at the various strain ranges. Table 2 displays the percentage change in stiffnesses of the various samples compared to control. This table demonstrates that the difference is larger at the lower strain ranges, for example Dresden and BS³-0.2M-15 min had >100% average increase in stiffness compared to the control at 5-10% strain, but at 30-35% strain the differences were 34% and 18% respectively.

TABLE 2
Percentage change in elastic modulus of ex vivo porcine corneas treated by
UV/riboflavin protocols or with BS³ compared to control calculated
at various strain ranges.
Percentage Change in Modulus Compared to Control

| | | | BS3- | BS3- | BS3- |
|---|---|---|---|---|---|
| Strain | | | 0.2M- | 0.2M- | 0.2M- |
| Range | Dresden | Accelerated | 5 min | 15 min | 30 min |
| 5-10% | 158% | 79% | 76% | 109% | 56% |
| 10-15% | 124% | 46% | 60% | 68% | 54% |
| 20-25% | 62% | 28% | 42% | 43% | 45% |
| 30-35% | 34% | 16% | 12% | 18% | 22% |

A formulation mentioned in US2014/0271897 A1 paragraph [0358] of 20 mg/ml (0.035 M) BS³ in PBS was also tested (FIG. 5). In this experiment no significant difference was seen compared to control p>0.05 at any of the strain ranges. The BS³ treatment had a reduction in stiffness until 20-25% strain (Table 3).

TABLE 3
Percentage change in elastic modulus of BS³ formulation from
US2014/0271897 A1 [0358] paragraph treated ex vivo porcine corneas
compared to control calculated at various strain ranges.
Percentage Change in Modulus Compared to Control

| Strain | 20 mg/ml |
|---|---|
| Range | BS3 |
| 5-10% | −28% |
| 10-15% | −10% |
| 20-25% | 3% |
| 30-35% | 15% |

### In Vivo Rat Studies

### Rat Corneal Thickness

The thicknesses of rat corneas were measured using OCT and compared to the contralateral control eye by paired t-test (FIG. 6). Immediately after treatment there was a significant reduction in corneal thickness in the treated eye of 42±29 μm (p=<0.0001). The thickness of the treated corneas normalised by 24 hours after treatment and there were no significant differences in corneal thickness 1 or 7 days after treatment.

### Rat Corneal Transparency

Rat corneal transparency was determined from the mean pixel intensity of corneal regions minus the background intensity. BS³ treated eyes were compared to contra-lateral control eyes immediately after treatment, at day 1, and day 7 (FIG. 7). No statistical differences were observed for any time point.

### Rat Histology

Histological examination of rat corneas at day 1 and day 7 after treatment with BS³ and the contralateral control eye showed no qualitative evidence of tissue toxicity. Large stratified epithelia were present on treated and controls and the stroma looked structurally similar (FIG. 8).

Similarly, no differences were observed between the control and treated tissues of lids and extraorbital lacrimal gland post-treatment (FIG. 9). Superior lids, day 1 post-treatment, display meibomian glands, stratified conjunctival epithelium and goblet cells. The inferior lids are similar structured without the observed goblet cells within the conjunctival epithelium, however micrographs of the inferior lids are of 7 days post-treatment as the conjunctiva did not survive the processing in the day 1 control and treated samples. Both control and treated extraorbital lacrimal glands are structurally similar with a high density of acini cells.

### Inflammatory Changes in the Aqueous Humour

A rat 12-plex xMAP-based cytokine assay was run on aqueous taken from treated and contra-lateral untreated control eyes. The assay determined the concentrations of IL-1α, IL-1β, IL-2, IL-4, IL-5, IL-6, IL-10, IL-12 (p70), IL-13 IFN-γ, GM-CSF and TNF-α. Due to the small volumes available aqueous of between 2-4 animals were pooled. The only statistically significant changes in cytokine levels between control and treated eyes were decreases observed in IL-2 and IL-4 at day 1 (FIGS. 10A and 10B). These reductions, −0.76 fold decrease in IL-2 and −0.63 fold decrease in IL-4, had resolved by day 7.

### In Vivo Rabbit Studies

### Rabbit Tensile Measurements

Treatment of the rabbit cornea with 0.2 M BS³ had insignificant effect on the stiffness of the corneal tissue in comparison with the control untreated eye at lower strain ranges after 3 days (5-10% & 10-15%, FIG. 11). At the 15-20% and 20-25% strain range BS³ treated was significantly stiffer (43% and 104%, respectively) than the contralateral controls (p<0.03).

Additional results after 28 days are provided in Table 5. 28 days after treatment a significant increase in stiffness was still present at strain ranges 10-15% and 15-20% (29% and 28% respectively) than contralateral controls. (p<0.04)

TABLE 4
Percentage change in elastic modulus of BS³ treated rabbit corneas after
3 days compared to control calculated at various strain ranges.
Percentage Change in Modulus After 3 Days Compared to Control

| Strain Range | Treated |
|---|---|
| 5-10% | −6% |
| 10-15% | 15% |
| 15-20% | 43% |
| 20-25% | 104% |

TABLE 5
Percentage change in elastic modulus of BS³ treated rabbit corneas after
28 days compared to control calculated at various strain ranges.
Percentage Change in Modulus Compared to Control

| Strain Range | Treated |
|---|---|
| 5-10% | 22% |
| 10-15% | 29% |
| 15-20% | 28% |
| 20-25% | 38% |

### Rabbit Corneal Thickness

The thicknesses of rabbit corneas were measured using OCT and compared to the contralateral control eye by paired t-test. 6 females and 5 males were tested and the mixed sex results are reported (FIG. 15 shows thickness after 3 days,). Immediately after treatment there was a significant reduction in corneal thickness in the treated eyes of 100±20 μm (p=<0.0001). There was still a slight reduction in corneal thickness at 3 days post-treatment of 11±14 μm (p=0.0332).

FIG. 16 shows rabbit corneal thickness after 28 days.

### Rabbit Corneal Transparency

Rabbit corneal transparency was determined from the mean pixel intensity of corneal regions minus the background intensity. BS³ treated eyes were compared to contra-lateral control eyes immediately after treatment and at day 3 (FIG. 13). There was a significant increase in opacity, i.e. increased pixel intensity, compared to control immediately after treatment (p>0.0001). By day 3 OCT pixel intensities were no longer significantly different (p=0.3309).

### Rabbit Histology

Histological examination of rabbit corneas at 3 days after treatment with BS³ and the contralateral control eye showed no qualitative evidence of tissue toxicity (FIG. 14). Stratified epithelia were present on treated and controls, the stroma appeared structurally similar, and an endothelium was present on most samples, although some separation from the Descemet's membrane does occur during processing (FIG. 14E).

1. A pharmaceutical composition suitable for administration to the eye, the pharmaceutical composition comprising:
(i) an aqueous vehicle; and
(ii) a water soluble cross-linker dissolved in the aqueous vehicle, wherein the water soluble cross-linker is a compound of Formula (I) shown below, or a pharmaceutically acceptable salt and/or solvate thereof; 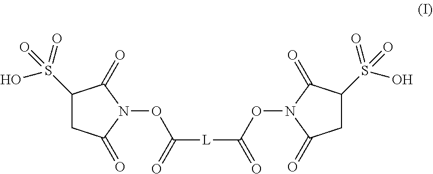
wherein L is a linear or branched (1-12C)alkylene linker that optionally comprises one or more heteroatoms selected from N, O or S, and is optionally substituted with one or more groups selected from carboxy, anhydride, oxo, halo, trifluoromethyl, cyano, nitro, hydroxy, mercapto, amino, (1-6C)alkyl, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, or (2-6C)alkanoyloxy;
and wherein the composition further comprises a buffer to maintain the pH within a range of 6.0 to 8.5 for at least 15 minutes.

2. The pharmaceutical composition according to claim 1, wherein L is a (2-8C)alkylene, and is optionally substituted with one or more groups selected from carboxy, anhydride, oxo, halo, trifluoromethyl, cyano, nitro, hydroxy, mercapto, amino, (1-4C)alkyl, (1-4)alkoxy, (1-4)alkylthio, (1-4C)alkylsulphinyl or (1-4C)alkylsulphonyl.

3. The pharmaceutical composition according to any one of the preceding claims, wherein L is a (5-7C)alkylene and is optionally substituted with one or more groups selected from carboxy, anhydride, oxo, halo, trifluoromethyl, cyano, nitro, hydroxy, mercapto and amino.

4. The pharmaceutical composition according to any one of the preceding claims, wherein L is a (5-7C)alkylene.

5. The pharmaceutical composition according to any one of the preceding claims, wherein the water soluble cross-linker is bissulfosuccinimidyl suberate, or a pharmaceutically acceptable salt and/or solvate thereof and optionally the disodium salt.

6. The pharmaceutical composition according to any one of the preceding claims, wherein the buffer is present in a concentration of from 0.05M to 2M, e.g. from 0.1 to 1M.

7. The pharmaceutical composition according to any one of the preceding claims, wherein the buffer is a carbonate-bicarbonate buffer.

8. The pharmaceutical composition according to any one of the preceding claims, wherein the buffer maintains the pH within a range of:
(i) 6.0 to 8.0, e.g. 6.5 to 8.0.

9. The pharmaceutical composition according to any one of the preceding claims, wherein the buffer maintains the pH in the stated range for:
a. greater than 30 minutes;
b. greater than 2 hours;
c. greater than 6 hours;
d. greater than 24 hours; or
e. greater than 48 hours.

10. The pharmaceutical composition according to any one of the preceding claims, wherein the water soluble cross-linker is present in an amount of 0.01 M to 0.3 M.

11. The pharmaceutical composition according to any one of the preceding claims, wherein the water soluble cross-linker is present in an amount of 0.1 M to 0.25 M.

12. A pharmaceutical composition according to any preceding claim for use in the treatment of a collagenic eye disorder.

13. A pharmaceutical composition for use according to claim 12, wherein the collagenic eye disorder is a disorder associated with the weakening, degradation and/or damage to collagen in the cornea and/or sclera of the eye.

14. A pharmaceutical composition for the use according to claim 12:
a) for use in the treatment of:
i. corneal ectasia, including: (i) non-inflammatory corneal ectasia—e.g. keratoconus, keratoglobus, pellucid marginal degeneration; (ii) inflammatory corneal ectasia; (iii) iatrogenic corneal ectasia (keratectasia); (iv) myopia;
ii. inflammation in the eye caused by infective, traumatic (chemical, physical, thermal, surgical) or immune-mediated (including vasculitic) corneal or scleral disease;
iii. re-shaping the eye, optionally following transplant or surgery; or
iv. corneal swelling due corneal oedema (e.g. bullous keratopathy, Fuchs Endothelial Dystrophy, Congenital Hereditary Endothelial Dystrophy, hydrops of the cornea in keratoconus);
or
b) for use in mechanically strengthening a weakened cornea and/or sclera in the treatment or prevention of:
i. myopia
ii. glaucoma
iii. microbial keratitis
iv. progressive diseases that cause change in size or thickness of the eye ball or its part (e.g. megalocornea, buphthalmos, peripheral ulcerative keratitis)
v. complications arising from refractive surgical procedures that weaken the cornea and/or sclera.

15. A pharmaceutical composition for use according to any one claims 12 to 14, for use in the treatment of keratoconus.

16. A pharmaceutical composition according to any one of claims 1 to 11 for use as a treatment in corneal transplant procedures to stiffen the cornea to make it more robust for suturing and/or to reduce the corneal astigmatism; or for use as a treatment in wound care to stiffen the tissue around a wound.

17. A method of treating a collagenic eye disorder, said method comprising administering to a human or animal subject in need of such treatment a therapeutically effective amount of a pharmaceutical composition according to any one of claims 1 to 11.

18. A method according to claim 17, wherein the collagenic eye disorder is a disorder associated with the weakening, degradation and/or damage to collagen in the cornea and/or sclera of the eye.

19. A method according to claim 17 or claim 18, wherein either:
(1) the collagenic eye disorder to be treated is selected from:
i. corneal ectasia, including: (i) non-inflammatory corneal ectasia—e.g. keratoconus, keratoglobus, pellucid marginal degeneration; (ii) inflammatory corneal ectasia; (iii) iatrogenic corneal ectasia (keratectasia); (iv) myopia;
ii. inflammation in the eye caused by infective, traumatic (chemical, physical, thermal, surgical) or immune-mediated (including vasculitic) corneal or scleral disease;
iii. re-shaping the eye, optionally following transplant or surgery; or
iv. corneal swelling due corneal oedema (e.g. bullous keratopathy, Fuchs Endothelial Dystrophy, Congenital Hereditary Endothelial Dystrophy, hydrops of the cornea in keratoconus); or
(2) the method involves mechanically strengthening a weakened cornea and/or sclera in the treatment or prevention of:
i. myopia
ii. glaucoma
iii. microbial keratitis
iv. progressive diseases that cause change in size or thickness of the eyeball or its part (e.g. megalocornea, buphthalmos, peripheral ulcerative keratitis)
v. complications arising from refractive surgical procedures that weaken the cornea and/or sclera.

20. A method according to any one of claims 17 to 19, wherein the collagenic eye disorder is keratoconus.

21. A method of corneal transplantation, wherein the method comprises treating the cornea of the eye in a patient in need of such treatment with a pharmaceutical composition according to any one of claims 1 to 11 to stiffen the cornea to make it more robust for suturing.

22. A device comprising a pharmaceutical composition according to any one of claims 1 to 11, wherein said device is configured to dispense a dose of the pharmaceutical composition to an eye of a patient.

23. A kit comprising:
a. an aqueous vehicle;
b. a water soluble cross-linker as defined in any one of claims 1 to 11; and
c. either:
a buffer comprised within the aqueous vehicle to maintain the pH within a range of 6.0 to 8.5 for at least 15 minutes after dissolving the water soluble cross-linker in the aqueous vehicle; or
a buffer solution, wherein the buffer maintains the pH within a range of 6.0 to 8.5 for at least 15 minutes after mixing with the water soluble cross-linker and aqueous vehicle with the buffer solution.
